(19) United States
(12) Patent Application Publication
(10) Pub. No.: US 2010/0056633 A1
(43) Pub. Date: Mar. 4, 2010

(54) SOLID OR AQUEOUS ALKALINE PREPARATION COMPRISING A CREATINE COMPONENT, PROCESS FOR THE PRODUCTION THEREOF AND THE USE THEREOF

(76) Inventor: Thomas Gastner, Engelsberg (DE)

Correspondence Address: FULBRIGHT & JAWORSKI, LLP, 666 FIFTH AVE, NEW YORK, NY, 10103-3198, US

(21) Appl. No.: 12/312,158

(22) PCT Filed: Oct. 26, 2007
(86) PCT No.: PCT/EP2007/009324

§ 371 Date: Apr. 28, 2009

(30) Foreign Application Priority Data
Oct. 28, 2006 (DE) 10 2006 050 931.5

(51) Int. Cl. A61K 31/195

(52) U.S. Cl. 514/565

(57) ABSTRACT

A solid or aqueous alkaline preparation comprising a creatine component which comprises a buffer system which adjusts a pH of from 8.0 to 12.0 is described. The creatine is better protected with the aid of the buffer system from conversion into creatinine in the stomach. It has additionally emerged, surprisingly, that the novel formulations display a distinctly higher bioavailability and are thus taken up better by cells. Finally, the preparation of the invention has very good organoleptic properties, which in fact likewise could not be predicted. Owing to these particular advantages, the preparation of the invention is outstandingly suitable as dietary supplements, restoratives, medicinal products and feedstuffs.

The present invention relates to a solid or aqueous alkaline preparation comprising a creatine component, to a process for the production thereof, and also to the use thereof as dietary supplements, restoratives, medicinal formulations and also feedstuffs.

In 1832, the French chemist Chevreul isolated a novel compound from meat broth. Cheuvreul named this compound creatine, using as a basis here the Greek word for meat (“Kreas”). This work was taken up again 15 years later by Justus von Liebig, who was able to show that creatine is a natural constituent of the muscle juice of vertebrates. The meat extract produced later by Liebig was the first commercially available food that contained creatine in concentrated form (approximately 10% by weight). At the time, in South America, there was a great excess of beef since it could not be transported over relatively long distances because of the lack of potential chilling methods. The animals were reared at that time especially for obtaining their hides, horns and bones. The discovery of the meat extract was a great market success since by this means the meat of the animals could also be expediently used. Later, the meat broth became also of great importance in Europe as a result of the wars and was used as a nutrient-rich strength-giving food for soldiers. The meat extract developed by Liebig is also popular still today for enriching the flavor of soups and sauces.

Since the end of the 1970s, the considered action of creatine was examined systematically. Up to now, over 300 studies have been carried out in the sports sector, wherein about 80% of these studies have demonstrated significant beneficial effects of creatine on muscle mass, muscle power, fat-free body mass and performance in various types of sports at maximum short-term muscle exertion. Creatine monohydrate is today the most important food supplement in the sports sector.

Only recently have further interesting properties of creatine become known. For instance, in two studies, significant beneficial effects of an oral creatine supplement on brain performance and concentration ability have been demonstrated (Rae, Caroline et al.: Oral creatine monohydrate supplementation improves brain performance: a double-blind, placebo-controlled, cross-over trial. Proceedings of the Royal Society of London, Series B: Biological Sciences (2003), 270(1529), 2147-2150; Watanabe, Airi et al.: Effects of creatine on mental fatigue and cerebral hemoglobin oxygenation. Neuroscience Research (Oxford, United Kingdom) (2002), 42(4), 279-285).

In addition, it has been found that creatine has antioxidative and neuroprotective properties and therefore can also be used for prevention of damage to cells due to environmental effects (Sestili, Piero et al.: Creatine supplementation affords cytoprotection in oxidatively injured cultured mammalian cells via direct antioxidant activity. Free Radical Biology & Medicine (2006), 40(5), 837-849; P. Klivenyi et al.: Neuroprotective effects of creatine in a transgenic animal model of amyotrophic lateral sclerosis. Nature Medicine 5, 347-350 (1999)). Creatine will therefore increase greatly in importance in the future in the anti-aging field also.

The beneficial effects of creatine are currently also being intensively studied in the medicinal field, wherein creatine is in the clinical phase 3 in the treatment of Parkinson's disease and amyotrophic lateral sclerosis (ALS) and in phase 2 in the case of Huntington's chorea (EP 804 183 B1). Successful use of creatine as a therapeutic agent against asthma has already also been reported (EP 911 026 B1). Creatine has shown beneficial effects in build-up of bones not only in vitro but also in vivo. The use for reinforcing bones and for treatment and prevention of degenerative bone and cartilage disorders such as, for instance, osteoporosis, has been studied and given very positive results (EP 1 100 488 B1; Gerber, I et al.: Stimulatory effects of creatine on metabolic activity, differentiation and mineralization of primary osteoblast-like cells in monolayer and micromass cell cultures. European Cells and Materials (2005), 10, 8-22; Chilibeck, P. D. et al.: Creatine monohydrate and resistance training increase bone mineral content and density in older men. Journal of Nutrition, Health & Aging (2005), 9(5), 352-355).

In addition, it is known that creatine supplementation leads to an increase in body mass. This is initially due to an increased uptake of water into the muscles. Viewed in the long term, creatine, however, leads indirectly by increased protein synthesis or reduced protein catabolism in the myofibrils to an increase in muscle mass (Int J Sports Med 21 (2000), 139-145). The result, therefore, is that an increased fat-free body mass is obtained.

However, in addition to creatine itself, that is creatine monohydrate, in the interim, numerous creatine salts such as creatine ascorbate, -citrate, -pyruvate, -phosphate and others, have likewise proved to be suitable food supplements. Representative examples which may be mentioned at this point as prior art are European patent EP 894 083 and German laid-open application DE 197 07 694 A1.

The metabolism and mode of action of creatine have been very well studied. Its biosynthesis proceeds from glycine and L-arginine. In mammals, especially in the kidneys, but also in the liver and pancreas, the guanidino group of L-arginine is cleaved by the enzyme aminotransferase and an N—C—N group is transferred to the glycine. The L-arginine in this case is converted into L-ornithine. The guanidino acetic acid thus formed is converted into creatine in the next step which, in vertebrates, proceeds chiefly in the liver, using the enzyme transmethylase. In this case the S-adenosylmethionine acts as methyl group donor. The creatine subsequently diffuses into the blood circulation and is thus transported to the target organs. Transport through the cell membrane into the cells proceeds in this case via a specific NaCl-dependent creatine transporter (Speer O, Neukomm L J, Murphy R M, Zanolla E, Schlattner U, Henry H, Snow R J, Wallimann T. Creatine transporters: a reappraisal. Mol Cell Biochem. 2004 January-February; 256-257(1-2):407-24).

Creatine plays an important role in the energy metabolism of cells, in which, as high-energy phosphocreatine, it is an essential energy reserve of muscle, in addition to adenosine triphosphate (ATP). In the resting state of the muscle, ATP can transfer a phosphate group to creatine, forming phosphocreatine which then is in direct equilibrium with ATP. During muscle work it is of critical importance to replenish the ATP stores as rapidly as possible. In the first seconds of maximum muscle strain, phosphocreatine is available for this. Phosphocreatine can, in a very rapid reaction via the enzyme creatine kinase, transfer a phosphate group to adenosine diphosphate, and thus reform ATP. This is also called the Lohmann reaction.

In addition, creatine has an important function in the transfer of energy in cells. What is termed the creatine shuttle system transports energy from the mitochondria to sites in the cell where the energy is required.

During muscle work which is vigorous and maintained over a relatively long time, the natural creatine stores present in the body are rapidly exhausted. For this reason, in particular in high-performance athletes, targeted creatine administration has given beneficial results on stamina and power, with unwanted accumulation processes in the body or disadvantageous breakdown products being unknown. The reason for this is considered to be that creatine is excreted from the body via the kidneys in the case of excess supply. In addition, creatine converts at a constant rate into the cyclic breakdown product creatinine, which is likewise excreted via the kidneys and thus is a second metabolic breakdown path.

The uptake of creatine into the musculature is controlled by an NaCl-dependent creatine transporter and can be beneficially influenced by the simultaneous uptake of carbohydrates and proteins. In this case it was found that the combination of creatine and carbohydrates, compared with intake of creatine alone, can lead to a 60% increased rise of the creatine content in muscles (Green A L, Hultman E, Macdonald I A, Sewell D A, Greenhaff P L. Carbohydrate ingestion augments skeletal muscle creatine accumulation during creatine supplementation in humans. Am J Physiol. 1996 November; 271 (5 Pt 1):E821-6). It was shown that the secretion of insulin during uptake of creatine into muscle cells plays an important role. There is a linear correlation between increase in creatine concentration in the musculature and the amount of insulin secreted (Steenge G R, Simpson E J, Greenhaff P L. Protein- and carbohydrate-induced augmentation of whole body creatine retention in humans. J Appl Physiol. 2000 September; 89(3):1165-71).

However, in addition to its uncontested beneficial physiological properties, creatine does have the disadvantage that it does not have pronounced stability in the corresponding aqueous solutions. Creatine cyclizes in this case by eliminating water to form creatinine. The cyclization rate is dependent on the pH of the solution and the temperature, with concentration not playing a role. Particularly in the neutral and acidic pH range, the conversion to creatinine proceeds very rapidly. Owing to the rapid breakdown of creatine in this environment, the use in aqueous or moist formulations for human and animal nutrition is virtually excluded. Just the pH of the stomach of 1 to 2 can, depending on residence time, lead to a significant breakdown of creatine to creatinine (Greenhaff, P. L.: Factors Modifying Creatine Accumulation in Human Skeletal Muscle. In: Creatine. From Basic Science to Clinical Application. Medical Science Symposia Series Volume 14, 2000, 75-82).

The stability of creatine as a function of pH was studied thoroughly as early as 1928 and the higher stability in the alkaline range has already been known since this time (Cannan, Robert Keith; Shore, Agnes. Creatine-creatinine equilibrium. The apparent dissociation constants of creatine and creatinine. Biochemical Journal (1928), 22, 920-9). The use of an alkaline creatine for preparations which are used for nutrition, however, was not described until much later.

For instance, EP 669 083 A2 claims an alkaline creatine drink and use thereof, which drink is distinguished by the stability of creatine during the preservation process. The scope of protection also extends here to a process in which 1.) water having a basic pH is charged and heated, 2.) 1-3 g of creatine per 100 ml are dissolved with stirring and 3.) additives for increasing the nutrient content and improving the flavor are added. A special base for setting the pH is not described in this application.

U.S. Pat. No. 6,399,661 claims a creatine preparation which is intended for nutritional purposes. The claimed production proceeds via a three-stage process in which 1.) an alkaline powder is mixed with pulverulent creatine in order to obtain a mixture of pH 7 to 14; 2.) a pulverulent additive is added in order to improve sweetness and taste of the mixture and 3.) a further alkaline powder is added in order to set the pH of the mixture to values between 7 and 14. The base used is preferably sodium carbonate and/or magnesium glycerolphosphate. In addition, the alkaline components can be selected from the group of hydroxides, carbonates, bicarbonates, chlorides, tree latex or phosphates.

EP 1 520 580 A1 claims a method of increasing the stamina in mammals and humans by using a creatine preparation which has a pH between 7 and 14. The preparations used correspond to the mixtures stipulated in U.S. Pat. No. 6,399,661.

A disadvantage with the preparations according to the prior art is the fact that even small amounts of acids are sufficient in order to neutralize these mixtures or set an acid pH. In practise, maximum dosages of some grams of such creatine preparations are selected. After dissolution in water, these are first stable, after oral uptake, such a dose, owing to the small amount of base present, is very rapidly set to an acidic pH by the stomach acid, and creatine is therefore unstable.

The basic creatine preparations which are known from the prior art are therefore not provided to the body in the maximum possible amount, since in addition, in the acid environment of the stomach, some of the creatine is converted to creatinine.

In the light of the disadvantages of the prior art described with respect to the stability of creatine, the object of the present invention was to develop preparations which protect the creatine better against breakdown to form creatinine in the stomach. A critical factor in this case is an optimal supply of the body cells with creatine without in this case creatinine being formed, which is of no use for the body and therefore must be excreted from the body via the kidneys.

This object has been achieved by providing a preparation comprising a creatine component, wherein the preparation, additionally to the creatine component, contains a buffer system which sets a pH of 8.0 to 12.0. In a preferred embodiment of the invention, the preparation is an alkaline preparation which, particularly preferably; is solid or aqueous.

It has been shown that using these formulations the objective could be achieved completely, namely protecting the creatine better by the buffer system against conversion to creatinine in the stomach. Surprisingly it has proved that the novel formulations have a significantly higher bioavailability and therefore can be taken up better into the cells. Furthermore, the preparation according to the invention has very good organoleptic properties which likewise could not have been predicted.

The preparation according to the present invention comprises a creatine component and a buffer system, wherein the buffer system is a combination of a weak acid and the conjugate base. The creatine component used is preferably creatine, creatine monohydrate and/or at least one salt and an addition compound or complex compound thereof. Particularly preferably, in the context of the present invention, the at least one salt, the at least one addition compound and/or complex compound is selected from the group consisting of malic acid, ascorbic acid, succinic acid, pyruvic acid, fumaric acid, aspartic acid, gluconic acid, α-ketoglutaric acid, oxalic acid, pyroglutamic acid, 3-nicotinic acid, maleic acid, sulfuric acid, acetic acid, formic acid, phosphoric acid, hydrochloric acid, 2-hydroxybenzoic acid, α-lipoic acid, L-carnitine, acetyl-L-carnitine, taurine, betaine, choline and methionine. In a preferred embodiment, the creatine component is present in solid form, particularly as powder, or in aqueous solution.

It is considered essential to the invention that the buffer system sets a pH of 8.0 to 12.0, and preferably 10.0 to 11.0. As preferred buffer system, the present invention envisages a mixture of sodium carbonate and sodium hydrogen carbonate. The ratio of the two components can be selected freely in broad ranges, wherein this is preferably selected in such a manner that the pH of the formulation establishes itself to 10.0 to 11.0. In this case it is advantageous that, with the correct choice of mixing ratio, the amount used is virtually unrestricted. For instance, when a 1 to 1 mixture is used a pH of 10.4 is inevitably established, wherein this is independent of the total amount of buffer used.

Therefore, a pH which is acceptable from the organoleptic aspect may be set and simultaneously the creatine is optimally protected against the influence of acid, as a result of which conversion to creatinine is avoided.

Further buffer systems which also come into consideration are mixtures of sodium hydrogen phosphate and sodium phosphate or L-lysine and L-lysine sodium salt or L-arginine and L-arginine sodium salt, wherein the ratio used is again selected in such a manner that the pH of the formulation preferably sets itself to 10.0 to 11.0.

The formulation is not limited with respect to the buffer component, wherein, in particular, the amount of the buffer component in which it can be present in the preparation is not a restriction. However, for nutritional reasons, amounts are recommended which are between 0.1 and 90.0% by weight, based on the total weight of the composition. Particular preference is given to amounts between 2.5 and 15.0% by weight, and in particular 5.0 to 10.0% by weight, based on the total weight of the preparation.

Surprisingly, in the use of the described buffer systems, it has proved that they lead not only to a lower breakdown of creatine in the stomach, but that the creatine administered is also taken up better into the cells. For instance, it was shown in an experiment that the formulations according to the invention lead to a significantly higher increase in the creatine concentrations in muscle than is the case with the use of creatine monohydrate or the known alkaline formulations according to the prior art.

In this connection it was found that the sodium content of the formulation has a decisive influence on the bioavailability and the uptake of creatine into the cells. This appears plausible owing to the dependence of the creatine transporter on sodium ions. The use of a mixture of creatine and sodium salts for improving the uptake of creatine into muscles has not hitherto been described and offers significant advantages compared with the previous practise of the use of high carbohydrate or protein dosages.

The present invention therefore envisages, in addition to the buffer system, also, optionally, the incorporation of one or more further physiologically acceptable sodium salts or a mixture thereof into the preparations according to the invention. Contemplated are, therefore, for example, sodium chloride, sodium sulfate, sodium acetate, sodium citrate, sodium gluconate, sodium ascorbate, sodium pantothenate and sodium lactate, or mixtures of these salts.

The fraction of these sodium salts is relatively uncritical, but it has proved to be particularly advantageous to use these further sodium salts in an amount of 0.1 to 75.0% by weight, in particular 5.0 to 55.0% by weight, and particularly preferably 10.0 to 20.0% by weight, based on the total weight of the preparation.

The use of the described buffer systems therefore appears to be ideal, since, firstly, the stability of the creatine to acids is increased and therefore the breakdown of creatine in the stomach is avoided. In addition, the sodium ions present improve the uptake into the cells, wherein this effect can also be further reinforced via the addition of further sodium salts.

According to a preferred embodiment, the preparation according to the invention contains further physiologically active compounds such as, for example, carbohydrates, fats, amino acids, proteins, vitamins, minerals, trace elements and also derivatives and mixtures thereof. In addition, for improvement of bioavailability, further α-lipoic acid and/or guanidinoacetic acid can be added to the preparation according to the invention. In the event that the preparation according to the invention is used as aqueous solution, the solids content is preferably set to 0.01 to 14.0% by weight.

The present invention further relates to a process for producing the preparation according to the invention, wherein the creatine component is charged, a buffer system, preferably a mixture of a weak acid and the conjugate base, is incorporated and, if appropriate, other sodium salts, physiologically active compounds and/or α-lipoic acid and/or guanidinoacetic acid are added. Preferably, the creatine component is charged as powder or aqueous solution. In addition, the buffer system is preferably incorporated homogeneously.

A preferred aspect of the invention relates to a pharmaceutical composition comprising the preparation according to the invention and also, if appropriate, one or more pharmaceutically acceptable carriers and/or auxiliaries. The present invention claims, in addition, the use of the preparation according to the invention as food supplement. In particular, the use of the claimed preparation as physiological restorative and, in this connection, in particular in the form of a functional food for humans is taken into account, wherein the sectors of schools, sports, reconvalescence and/or geriatrics are in the foreground.

The described beneficial effects are also developed by the formulations described in animals, so that the use in this sector is also envisaged. If the creatine formulations described are used as feedstuff additive, in particular the administration to breeding animals and growing animals and also to animals in high-performance sport is considered as preferred, and in this context, particularly preferably to hogs, horses, poultry and fish, wherein the use as substitute for animal meal and/or fish meal and also products produced therefrom has proved to be particularly suitable. The replacement can in this case be a partial or complete replacement.

In addition, the novel creatine preparation can also be used as dietary supplement or nutritional constituent for domestic animals such as dogs, cats and birds.

As application forms, particularly powders, granules, pastils, capsules, tablets, solutions, juices and/or jelly products have proved to be particularly suitable.

In this case, depending on the respective specific application case, it can be thoroughly advisable to use the preparation according to the invention in combination with other physiologically active ingredients.

The preparation according to the invention can be administered in single doses of 0.001 to 0.3 g/kg of body weight, and in daily doses, which are between 0.001 and 1.0 g/kg of body weight, respectively. This applies, in particular, to the pharmaceutical composition and also to the use as feedstuff, dietary supplement, physiological restorative, but also as functional food.

Overall, the proposed formulation and use thereof are a further advance of the prior art with respect to increasing the stability of creatine formulations. Furthermore, improved bioavailability of the creatine component proved to be particularly advantageous.

The examples hereinafter illustrate the advantages of the present invention.

### EXAMPLES

### 1. Dietary Supplements

Hereinafter, typical compositions of neutral or good-tasting formulations are listed, the constituents of which are introduced at room temperature into 500 ml of fruit juice, water, yogurt and/or whey.

| | | |
|---|---|---|
| 1.1 | 2980 mg | Creatine monohydrate |
| | 150 mg | Sodium carbonate |
| | 118 mg | Sodium hydrogen carbonate |
| 1.2 | 1500 mg | Creatine monohydrate |
| | 400 mg | Sodium carbonate |
| | 600 mg | Sodium hydrogen carbonate |
| | 100 mg | Sodium citrate |
| | 2000 mg | Sodium chloride |
| 1.3 | 1500 mg | Creatine monohydrate |
| | 4000 mg | Sodium carbonate |
| | 6000 mg | Sodium hydrogen carbonate |
| | 500 mg | Guanidinoacetic acid |
| | 500 mg | Betaine |
| | 300 mg | α-Lipoic acid |
| | 400 mg | (MgCO₃)₄•Mg(OH)₂•5H₂O = approx. 100 Mg |
| | 500 mg | Vitamin C |
| 1.4 | 1500 mg | Creatine monohydrate |
| | 750 mg | L-arginine |
| | 250 mg | L-arginine sodium salt |
| | 1000 mg | Glucosamine |
| | 300 mg | Chondroitine sulfate |
| | 500 mg | Methionine |
| | 3100 mg | Creatinole sulfate |
| 1.5 | 750 mg | Creatine monohydrate |
| | 750 mg | L-lysine |
| | 750 mg | L-lysine sodium salt |
| | 1000 mg | Sodium ascorbate |

### 2. Feedstuffs

2.1 A formulation comprising 2000 mg of creatine citrate, 5000 mg of inuline, 3000 mg of sodium chloride, 600 mg of sodium carbonate and 700 mg of sodium hydrogen carbonate were introduced into a typical formula for feed pellets for feed supplementation of horses.
2.2 A formulation comprising 7000 mg of creatine monohydrate, 750 mg of carnitine tartrate, 100 mg of succrose stearate, 160 mg of talcum, 1090 mg of fructose, 2000 mg of sodium carbonate and 4700 mg of sodium hydrogen carbonate was introduced into the base mass for dog biscuits.
2.3 As a master batch, to a commercially available tinned cat food mixture, the following formulation was introduced homogeneously: 3000 mg of creatinole sulfate, 3000 mg of creatine monohydrate, 40 mg of magnesium stearate, 25 mg of carboxymethylcellulose and 135 mg of lactose, 500 mg of sodium phosphate and 1500 mg of sodium hydrogen phosphate.

### 3. Behavior to Acids

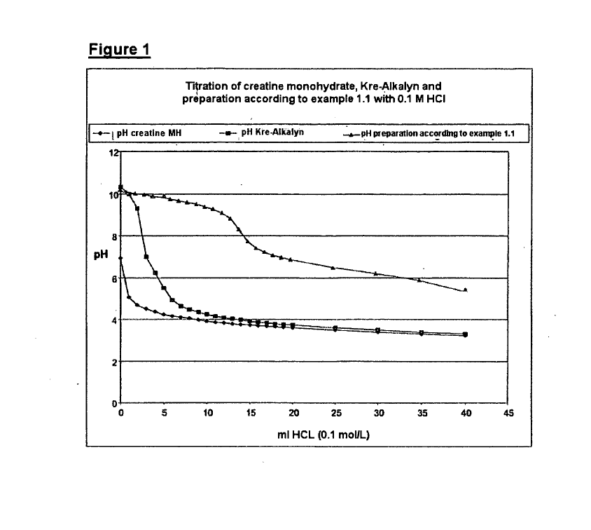

The effect of the addition of a strong acid to the pH of a solution of a creatine preparation according to the invention was studied and compared with the alkaline creatine preparations (Kre-Alkalyn®) available on the market up to now and creatine monohydrate: Creatine monohydrate and Kre-Alkalyn® and a creatine preparation according to the invention of example 1.1 were each dissolved in 500 ml of water. The amount was always selected such that in each solution 2980 mg of creatine monohydrate were introduced. Subsequently, the sample was titrated with 0.1 molar hydrochloric acid, wherein the pH course was measured using a pH electrode. The formulation according to the invention tolerated in this case a significantly larger addition of acid before it turned over to the acid range, as can clearly be seen from FIG. 1.

### Bioavailability

Three groups of testers, each of ten people, were assembled such that in all groups approximately the same mean starting values of creatine in muscle dry mass were present.

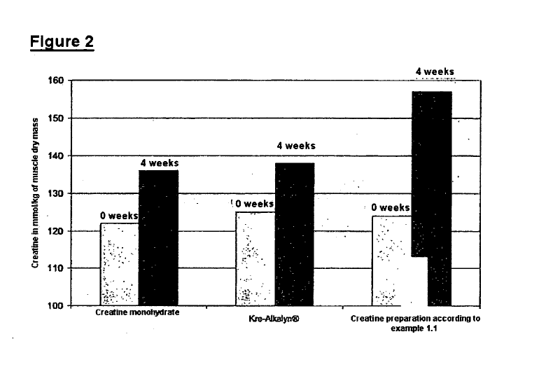

Over four weeks, a preparation according to the invention as per example 1.1, creatine monohydrate, or Kre-Alkalyn® was administered daily to the three groups. The dose in this case was selected such that per day, in each case 2.0 g of pure creatine monohydrate was taken up by each tester. Immediately before the study and two weeks after intake, the creatine content was measured in the muscle by means of muscle biopsy. The results are shown in FIG. 2.

1-19. (canceled)

20. A composition comprising (a) a creatine component, and (b) a buffer system comprising a combination of a weak acid and a conjugate base, selected from the group consisting of a sodium carbonate/sodium hydrogen carbonate, a sodium phosphate/sodium hydrogen phosphate, an L-lysine/L-lysine sodium salt and an L-arginine/L-arginine sodium salt, wherein said composition has a pH of from 8.0 to 12.0.

21. The composition of claim 20, wherein said creatine component is creatine, a creatine monohydrate, a salt, an addition compound, or a complex compound.

22. The composition of claim 21, wherein said salt, said addition compound, or said complex compound is selected from the group consisting of malic acid, ascorbic acid, succinic acid, pyruvic acid, fumaric acid, aspartic acid, gluconic acid, α-ketoglutaric acid, oxalic acid, pyroglutamic acid, 3-nicotinic acid, maleic acid, sulfuric acid, acetic acid, formic acid, phosphoric acid, hydrochloric acid, 2-hydroxybenzoic acid, α-lipoic acid, L-carnitine, acetyl-L-carnitine, taurine, betaine, choline and methionine.

23. The composition of claim 20, wherein said buffer system is present in an amount by weight relative to total weight of the composition of from 0.1 to 90.0%.

24. The composition of claim 20, wherein said composition further comprises at least one sodium salt.

25. The composition of claim 24, wherein said sodium salt is selected from the group consisting of sodium chloride, sodium sulphate, sodium acetate, sodium citrate, sodium gluconate, sodium ascorbate, sodium pantothenate and sodium lactate.

26. The composition of claim 24, wherein said sodium salt is present in an amount by weight relative to total weight of the composition of from 0.1 to 75.0%.

27. The composition of claim 20, further comprising a compound selected from the group consisting of a carbohydrate, a fat, an amino acid, a protein, a vitamin, a mineral, a trace element and a derivative thereof.

28. The composition of claim 20, further comprising a guanidinoacetic acid.

29. The composition of claim 20, further comprising an α-lipoic acid.

30. The composition of claim 20, further comprising a guanidinoacetic acid and an α-lipoic acid.

31. The composition of claim 20, wherein said composition has a pH of from 10.0 to 11.0.

32. The composition of claim 20, wherein said composition is solid or aqueous.

33. The composition of claim 20, wherein said composition is aqueous and said aqueous composition has a solids content of 0.01 to 14.0% by weight, based on the total weight of the composition.

34. The composition of claim 20, wherein said composition is a powder, a granule, a pastil, a capsule, a tablet, a solution, a juice or a jelly product.

35. The composition of claim 20, further comprising a pharmaceutically acceptable carrier or an auxiliary agent.

36. A method for reducing conversion of creatine to creatinine in a subject, comprising administering the composition of claim 20 to a subject in need thereof, at a single dose of from 0.001 to 0.3 g/kg of body weight, wherein said administration increases uptake of creatine into cells of said subject.

37. A method for reducing conversion of creatine to creatinine in a subject, comprising administering the composition of claim 20 to a subject in need thereof, at a daily dose of from 0.001 to 1 g/kg of body weight, wherein said administration increases uptake of creatine into cells of said subject.

38. A process for producing the composition of claim 20, comprising admixing a creatine component and a buffer system comprising a combination of a weak acid and a conjugate base, selected from the group consisting of a sodium carbonate/sodium hydrogen carbonate, a sodium phosphate/sodium hydrogen phosphate, an L-lysine/L-lysine sodium salt and an L-arginine/L-arginine sodium salt.

39. The process of claim 38, further comprising adding at least one sodium salt.

40. The process of claim 38, further comprising adding a compound selected from the group consisting of a carbohydrate, a fat, an amino acid, a protein, a vitamin, a mineral, a trace element and a derivative thereof.

41. The process of claim 38, further comprising adding a guanidinoacetic acid.

42. The process of claim 38, further comprising adding an α-lipoic acid.

43. The process of claim 38, further comprising adding a guanidinoacetic acid and an α-lipoic acid.
